(19) United States
(12) Patent Application Publication
(10) Pub. No.: US 2012/0157711 A1
(43) Pub. Date: Jun. 21, 2012

(54) PROCESS FOR PREPARING FORMIC ACID BY REACTING CARBON DIOXIDE WITH HYDROGEN

(75) Inventors: Thomas Schaub, Neustadt (DE); Donata Maria Fries, Mannheim (DE); Rocco Paciello, Bad Durkheim (DE); Klaus-Dieter Mohl, Hockenheim (DE); Martin Schäfer, Grunstadt (DE); Stefan Rittinger, Mannheim (DE); Petra Deckert, Bammental (DE); Peter Bassler, Viernheim (DE)

(73) Assignee: BASF SE, Ludwigshafen (DE)

(21) Appl. No.: 13/330,974

(22) Filed: Dec. 20, 2011

Related U.S. Application Data
(60) Provisional application No. 61/425,380, filed on Dec. 21, 2010.

(51) Int. Cl. C07C 53/02

(52) U.S. Cl. 562/609

(57) ABSTRACT

The present invention relates to a process for preparing formic acid by reacting carbon dioxide (1) with hydrogen (2) in a hydrogenation reactor (I) in the presence of
a catalyst comprising an element of group 8, 9 or 10 of the Periodic Table,
a tertiary amine comprising at least 12 carbon atoms per molecule and
a polar solvent comprising one or more monoalcohols selected from among methanol, ethanol, propanols and butanols,
to form formic acid/amine adducts as intermediates which are subsequently thermally dissociated,
where a tertiary amine having a boiling point which is at least 5° C. higher than that of formic acid is used and
a reaction mixture comprising the polar solvent, the formic acid/amine adducts, the tertiary amine and the catalyst is formed in the reaction in the hydrogenation reactor (I) and is discharged from the reactor as output (3).

This patent application claims the benefit of pending U.S. provisional patent application Ser. No. 61/425,380 filed Dec. 21, 2010 incorporated in its entirety herein by reference.

The invention relates to a process for preparing formic acid by reacting carbon dioxide with hydrogen in a hydrogenation reactor in the presence of a catalyst comprising an element of group 8, 9 or 10 of the Periodic Table, a tertiary amine and a polar solvent to form formic acid/amine adducts as intermediates which are subsequently thermally dissociated.

Adducts of formic acid and tertiary amines can be thermally dissociated into free formic acid and tertiary amine and therefore serve as intermediates in the preparation of formic acid.

Formic acid is an important and versatile product. It is used, for example, for acidification in the production of animal feeds, as preservative, as disinfectant, as assistant in the textile and leather industry, as mixture with its salts for deicing aircraft and runways and also as synthetic building block in the chemical industry.

The abovementioned adducts of formic acid and tertiary amines can be prepared in various ways, for example (i) by direct reaction of the tertiary amine with formic acid, (ii) by hydrolysis of methyl formate to formic acid in the presence of the tertiary amine or (iii) by catalytic hydrogenation of carbon monoxide or hydrogenation of carbon dioxide to formic acid in the presence of the tertiary amine. The latter process of catalytic hydrogenation of carbon dioxide has the particular advantage that carbon dioxide is available in large quantities and is flexible in terms of its source.

The catalytic hydrogenation of carbon dioxide in the presence of amines (W. Leitner, Angewandte Chemie 1995, 107, pages 2391 to 2405; P. G. Jessop, T. lkariya, R. Noyori, Chemical Reviews 1995, 95, pages 259 to 272) appears to be especially promising from an industrial point of view. The adducts of formic acid and amines formed here can be thermally dissociated into formic acid and the amine used, which can be recirculated to the hydrogenation.

The catalyst required for the reaction comprises one or more elements from group 8, 9 or 10 of the Periodic Table, i.e. Fe, Co, Ni, Ru, Rh, Pd, Os, Ir and/or Pt. The catalyst preferably comprises Ru, Rh, Pd, Os, Ir and/or Pt, particularly preferably Ru, Rh and/or Pd and very particularly preferably Ru.

To make an economical process possible, the catalyst used has to be separated off ideally completely from the product stream and recirculated to the reaction, for two reasons:
(1) large losses of the expensive catalyst would incur considerable additional costs and would be prohibitive for economical operation of the process.
(2) very little catalyst should be present in the thermal dissociation of the formic acid/amine adducts since it would, in the absence of a CO₂ and/or H₂ pressure, also catalyze the backreaction and thus lead to losses of the formic acid formed.

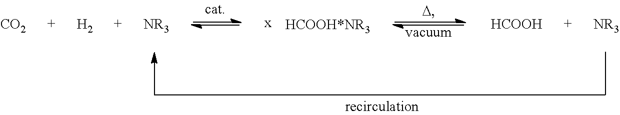

Formation of formic acid by hydrogenation of CO₂ (x=0.4−3)

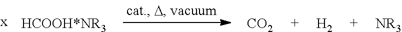

Decomposition of formic acid/amine adducts in the presence of a catalyst (x=0.4−3)

The transition metal-catalyzed decomposition of formic acid has been described in detail, especially recently: C. Fellay, N. Yan, P. J. Dyson, G. Laurenczy Chem. Eur. J. 2009, 15, 3752-3760; C. Fellay, P. J. Dyson, G. Laurenczy Angew. Chem. 2008, 120, 4030-4032; B. Loges, A. Boddien, H. Junge, M. Beller Angew. Chem. 2008, 120, 4026-4029; F. Joó Chem Sus Chem 2008, 1, 805-808; S. Enthaler Chem Sus Chem 2008, 1, 801-804; S. Fukuzumi, T. Kobayashi, T. Suenobu Chem Sus Chem 2008, 1, 827-834;A. Boddien, B. Loges, H. Junge, M. Beller Chem Sus Chem 2008, 1, 751-758.

The catalysts used here are in principle also suitable for the hydrogenation of CO₂ to formic acid (P. G. Jessop, T. Ikariya, R. Noyori Chem. Rev. 1995, 95, 259-272; P. G. Jessop, F. Joó, C. C. Tai Coord. Chem. Rev. 2004, 248, 2425-2442; P. G. Jessop, Homogeneous Hydrogenation of Carbon Dioxide, in: The Handbook of Homogeneous Hydrogenation, editor: J. G. de Vries, C. J. Elsevier, Volume 1, 2007, Wiley-VCH, pp. 489-511). Thus, the hydrogenation catalysts have to be separated off before the thermal dissociation in order to prevent the undesirable decomposition of formic acid.

WO 2008/116799 discloses a process for hydrogenating carbon dioxide in the presence of a catalyst which comprises a transition metal of transition group VIII (groups 8, 9, 10) and is suspended or homogeneously dissolved in a solution, a tertiary amine having at least one hydroxyl group and a polar solvent to give an adduct of formic acid and the tertiary amine. As a result of the hydroxyl group(s) in the tertiary amine, an increased, compared to the triethylamine which is otherwise usually used, carbon dioxide solubility is achieved. RuH₂L₄ having monodentate phosphorus-based ligands L and RuH₂(LL)₂ having bidentate phosphorus-based ligands LL and, as particularly preferred, RuH₂[P(C₆H₅)₃]₄ are mentioned as preferred homogeneous catalysts. As polar solvents, mention is made of alcohols, ethers, sulfolanes, dimethyl sulfoxide and amides whose boiling point at atmospheric pressure is at least 5° C. above that of formic acid. The tertiary amines which are preferably used also have a boiling point above that of formic acid. Since no phase separation takes place, the total reaction output is worked up by distillation, optionally after prior removal of the catalyst, with the resulting adduct of formic acid and the tertiary amine being thermally dissociated and the formic acid liberated being obtained as overhead product. The bottom product comprising tertiary amine, polar solvent and possibly catalyst is recirculated to the hydrogenation stage.

A disadvantage of this process is transfer of the entire liquid reaction output into the apparatus for thermal dissociation and distillation, optionally after prior specific removal of the homogeneous catalyst by means of a separate process step such as an extraction, adsorption or ultrafiltration stage. A consequence of this is that the apparatus for the thermal dissociation and distillation has to be made larger and more complex because of the higher liquid loading and also because of the more specific separation properties, which, inter alia, is reflected in the capital costs (for example via engineering input, material, space requirement). In addition, the higher liquid loading also results in a higher energy consumption.

However, the fundamental studies on the catalytic hydrogenation of carbon dioxide to formic acid were carried out as early as the 1970s and 1980s. The processes of BP Chemicals Ltd patented in EP 0 095 321 A, EP 0 151 510 A and EP 0 181 078 A may well have arisen therefrom. All three documents describe the hydrogenation of carbon dioxide in the presence of a homogeneous catalyst comprising a transition metal of transition group VIII (groups 8, 9, 10), a tertiary amine and a polar solvent to form an adduct of formic acid and the tertiary amine. As preferred homogeneous catalysts, EP 0 095 321 A and EP 0 181 078 A each mention ruthenium-based carbonyl-, halide- and/or triphenylphosphine-comprising complex catalysts and EP 0 151 510 A mentions rhodium-phosphine complexes. Preferred tertiary amines are C₁-C₁₀-trialkylamines, in particular the short-chain C₁-C₄-trialkylamines, and cyclic and/or bridged amines such as 1,8-diazabicyclo[5.4.0]undec-7-ene, 1,4-diazabicyclo[2.2.2]octane, pyridine or picolines. The hydrogenation is carried out at a carbon dioxide partial pressure of up to 6 MPa (60 bar), a hydrogen partial pressure of up to 25 MPa (250 bar) and a temperature of from about room temperature to 200° C.

EP 0 095 321 A and EP 0 151 510 A teach the use of an alcohol as polar solvent. However, since primary alcohols tend to form formic esters (organic formates), secondary alcohols, in particular isopropanol, are preferred. In addition, the presence of water is said to be advantageous. According to the examples of EP 0 095 321 A, the reaction output is worked up by a directly subsequent two-stage distillation in which the low boilers, alcohol, water, tertiary amine are separated off overhead in the first stage and the adduct of formic acid and the tertiary amine are separated off overhead under vacuum conditions in the second stage. EP 0 151 510 A likewise teaches a work-up by distillation, but with reference to EP 0 126 524 A with subsequent replacement of the tertiary amine in the adduct which has been separated off by distillation by a weaker, less volatile nitrogen base before thermal dissociation of the adduct in order to aid, or even make possible, the subsequent thermal dissociation to prepare the free formic acid.

EP 0 181 078 A teaches the targeted selection of the polar solvent on the basis of three main criteria which have to be fulfilled simultaneously:
(i) the homogeneous catalyst has to be soluble in the polar solvent;
(ii) the polar solvent must not have an adverse effect on the hydrogenation; and
(iii) the resulting adduct of formic acid and the tertiary amine should be able to be separated off easily from the polar solvent.

As particularly suitable polar solvents, mention is made of various glycols and phenylpropanols.

According to the teaching of EP 0 181 078 A, the reaction output is worked up by firstly separating off the gaseous components (first and foremost unreacted starting materials hydrogen and carbon dioxide) at the top of an evaporator and separating off the homogeneous catalyst dissolved in the polar solvent at the bottom and recirculating them to the hydrogenation stage. The adduct of formic acid and the tertiary amine is then separated off from the remaining liquid phase comprising the adduct of formic acid and tertiary amine, free tertiary amine and possibly water and the remaining part of the liquid phase comprising the free tertiary amine and possibly water is recirculated to the hydrogenation stage. The separation can be effected by distillation or by phase separation of the two-phase system (decantation).

A further important teaching from EP 0 181 078 A is the subsequent, compulsory replacement of the tertiary amine in the isolated adduct by a weaker, less volatile nitrogen base before thermal dissociation of the adduct in order to aid, or even make possible, the subsequent thermal dissociation to prepare the free formic acid. Imidazole derivatives such as 1-n-butylimidazole are mentioned as particularly suitable weaker nitrogen bases.

A disadvantage of the process of EP 0 181 078 A is the very complicated four-stage work-up of the reaction output by
(i) separating off the gaseous components and the homogeneous catalyst and the polar solvent in an evaporator and recirculating them to the hydrogenation stage;
(ii) separating off the adduct of formic acid and the tertiary amine in a distillation column or a phase separator and recirculating the remaining liquid stream to the hydrogenation stage;
(iii) replacing the tertiary amine in the adduct of formic acid and the tertiary amine by a weaker, less volatile nitrogen base in a reaction vessel with superposed distillation column and recirculating the liberated tertiary amine to the hydrogenation stage; and
(iv) thermally dissociating the adduct of formic acid and the weaker nitrogen base and recirculating the liberated weaker nitrogen base to the base replacement stage.

A further, important disadvantage of the process of EP 0 181 078 A, and also of the processes of EP 0 095 321 A and EP 0 151 510 A, is the fact that the adduct of formic acid and the tertiary amine partly redissociates into carbon dioxide and hydrogen in the presence of the homogeneous catalyst in the work-up in the evaporator. In EP 0 329 337 A, the addition of a decomposition inhibitor which reversibly inhibits the homogeneous catalyst is therefore proposed as a solution to the problem. As preferred decomposition inhibitors, mention is made of carbon monoxide and oxidants. However, disadvantages of this are introduction of further substances into the total process and the necessity of reactivating the inhibited homogeneous catalyst before it is used again.

EP 0 357 243 A also addresses the disadvantage of partial redissociation of the adduct of formic acid and the tertiary amine in the process of EP 0 181 078 A by joint work-up of the reaction output in the evaporator. The process proposed in EP 0 357 243 A teaches the use of a homogeneous catalyst comprising a transition metal of transition group VIII (groups 8, 9, 10), a tertiary amine and two different solvents, namely a nonpolar solvent and a polar solvent, in each case inert, which form two immiscible liquid phases in the catalytic hydrogenation of carbon dioxide to form an adduct of formic acid and a tertiary amine. As nonpolar solvents, mention is made of aliphatic and aromatic hydrocarbons, but also phosphines having aliphatic and/or aromatic hydrocarbon radicals. Polar solvents mentioned are water, glycerol, alcohols, polyols, sulfolanes or mixtures thereof, with water being preferred. The homogeneous catalyst dissolves in the nonpolar solvent, and the adduct of formic acid and the tertiary amine dissolves in the polar solvent. After the reaction is complete, the two liquid phases are separated, for example by decantation, and the nonpolar phase comprising the homogeneous catalyst and the nonpolar solvent is recirculated to the hydrogenation stage. The polar phase comprising the adduct of formic acid and the tertiary amine and the polar solvent is then subjected to a compulsory replacement of the tertiary amine in the adduct by a weaker, less volatile nitrogen base before the adduct is thermally dissociated in order to aid, or even make possible, the subsequent thermal dissociation to prepare free formic acid. Here too, imidazole derivatives such as 1-n-butylimidazole are, in a manner analogous to EP 0 181 078 A, mentioned as particularly suitable weaker nitrogen bases.

A disadvantage of the process of EP 0 357 243 A is the very complicated, three-stage work-up of the reaction mixture by
(i) separating the two liquid phases and recirculating the phase comprising the homogeneous catalyst and the nonpolar solvent to the hydrogenation stage;
(ii) replacing the tertiary amine in the adduct of formic acid and the tertiary amine of the other phase by a weaker, less volatile nitrogen base in a reaction vessel with superposed distillation column and recirculating the liberated tertiary amine to the hydrogenation stage; and
(iii) thermally dissociating the adduct of formic acid and the weaker nitrogen base and recirculating the liberated weaker nitrogen base to the base replacement stage.

A further disadvantage of the process of EP 0 357 243 A is the use of two solvents and thus introduction of a further substance into the total process.

As an alternative, EP 0 357 243 A also discloses the possibility of using only one solvent. In this case, the addition of the polar solvent in which the adduct of formic acid and the tertiary amine would otherwise dissolve is omitted. The only solvent used here is the nonpolar solvent which dissolves the homogeneous catalyst. However, this alternative also has the disadvantage of the very complicated, three-stage work-up as described above.

DE 44 31 233 A likewise describes the hydrogenation of carbon dioxide in the presence of a catalyst comprising a transition metal of transition group VIII (groups 8, 9, 10), a tertiary amine and a polar solvent and water to form an adduct of formic acid and the tertiary amine, in which, however, the catalyst is present in heterogeneous form and the active component is applied to an inert support. Preferred tertiary amines are C₁-C₈-trialkylamines, polyamines having from 2 to 5 amino groups, aromatic nitrogen heterocycles such as pyridine or N-methylimidazole and also cyclic and/or bridged amines such as N-methylpiperidine, 1,8-diazabicyclo[5.4.0]undec-7-ene or 1,4-diazabicyclo[2.2.2]octane. As suitable polar solvents, mention is made of the low-boiling C₁-C₄-monoalcohols, with secondary alcohols being preferred, in a manner analogous to EP 0 095 321 A. The hydrogenation is carried out at a total pressure of from 4 to 20 MPa (from 40 to 200 bar) and a temperature of from 50 to 200° C. To work up the resulting adduct of formic acid and tertiary amine, DE 44 31 233 A teaches the use of known methods with explicit reference to the work-up with replacement of the tertiary amine in the adduct of formic acid and the tertiary amine by a weaker, less volatile nitrogen base, as disclosed in EP 0 357 243 A. In the process according to DE 44 31 233 A, too, the very complicated, three-stage work-up of the reaction output is also disadvantageous, in a manner analogous to the process of EP 0 357 243 A.

It was an object of the present invention to provide a process for preparing formic acid by hydrogenating carbon dioxide, which does not have the abovementioned disadvantages of the prior art or has them only to a significantly reduced extent and leads to concentrated formic acid in high yield and high purity. Furthermore, the process should allow simple or at least simpler operation than that described in the prior art, in particular a simpler process concept for working up the output from the hydrogenation reactor, simpler process stages, a smaller number of process stages or simpler apparatuses. In addition, the process should also be able to be carried out with a very low energy consumption. In particular, an efficient solution to the previously only unsatisfactorily solved problem of recirculation of the catalyst while simultaneously ensuring a high activity of the hydrogenation catalyst should be offered.

The work-up of the output from the hydrogenation reactor should, in particular, be carried out using exclusively the materials already present in the process, without additional auxiliaries, and these should be recycled completely or largely completely in the process.

The object is achieved by a process for preparing formic acid by reacting carbon dioxide (1) with hydrogen (2) in a hydrogenation reactor (I) in the presence of
a catalyst comprising an element of group 8, 9 or 10 of the Periodic Table,
a tertiary amine comprising at least 12 carbon atoms per molecule and
a polar solvent comprising one or more monoalcohols selected from among methanol, ethanol, propanols and butanols,

to form formic acid/amine adducts as intermediates which are subsequently thermally dissociated,

where a tertiary amine having a boiling point which is at least 5° C. higher than that of formic acid is used and

a reaction mixture comprising the polar solvent, the formic acid/amine adducts, the tertiary amine and the catalyst is formed in the reaction in the hydrogenation reactor (I) and is discharged from the reactor as output (3),

wherein

the output (3) from the hydrogenation reactor (I) is, optionally after addition of water, fed directly to an extraction unit (II) and the work-up of the output (3) comprises the following process steps:
1) extraction of the catalyst from the output (3) from the hydrogenation reactor (I) in the extraction unit (II), where the same tertiary amine which was used in the hydrogenation is used as extractant, to give an extract (4) which comprises the major part of the tertiary amine and the catalyst and is recycled to the hydrogenation reactor (I) and also a raffinate (5) which comprises the major part of the polar solvent and the formic acid/amine adducts and is passed on to a distillation unit (III) in which
2) a distillation is carried out to give an overhead stream (6) which comprises predominantly the polar solvent and is recycled to the hydrogenation reactor (I) and also a bottom stream (7) which comprises predominantly the formic acid/amine adducts and the tertiary amine and is according to process step
3) fed to a phase separation vessel (IV) in which
a) a phase separation is carried out to give an upper phase which comprises predominantly the tertiary amine and is recycled as stream (11) to the extraction unit (II) as extractant for the catalyst and a lower phase which comprises predominantly the formic acid/amine adducts and is fed as stream (8) to a thermal dissociation unit (V) in which
b) a thermal dissociation is carried out to give a stream (9) which comprises the tertiary amine and is recirculated to the phase separation vessel (IV) and a stream (10) comprising the formic acid
   or according to process step
4) is fed to a thermal dissociation unit (V) in which
a) a thermal dissociation is carried out to give a stream (10) comprising the formic acid and a stream (9) which comprises the tertiary amine and the formic acid/amine adducts and is fed to a phase separation vessel (IV) in which
b) a phase separation is carried out to give an upper phase which comprises predominantly the tertiary amine and is recycled as stream (11) to the extraction unit (II) as extractant for the catalyst and a lower phase which comprises predominantly the formic acid/amine adducts and is fed as stream (8) to a thermal dissociation unit (V).

In an embodiment of the present invention, only a substream of the tertiary amine-comprising stream (11) from the phase separation vessel (IV) is introduced into the extraction unit (II) as selective extractant for the catalyst and the remaining part of the tertiary amine-comprising stream is introduced directly into the hydrogenation reactor (I).

For the purposes of the present invention, “fed directly to an extraction unit (II)” means that the output (3) from the hydrogenation reactor (I) is, optionally after addition of water, fed without further work-up steps to an extraction unit (II).

In one embodiment, the process of the invention therefore comprises the process steps 1), 2) and 3). In a further embodiment, the process of the invention comprises the steps 1), 2) and 4).

The catalyst used in the hydrogenation of carbon dioxide in the process of the invention is preferably a homogeneous catalyst. It comprises an element of group 8, 9 or 10 of the Periodic Table (in the IUPAC version), i.e. Fe, Co, Ni, Ru, Rh, Pd, Os, Ir and/or Pt. The catalyst preferably comprises Ru, Rh, Pd, Os, Ir and/or Pt, particularly preferably Ru, Rh and/or Pd, and very particularly preferably Ru.

The elements mentioned are present in the form of complexes homogeneously dissolved in the reaction mixture. The homogeneous catalyst should be selected so that it accumulates together with the tertiary amine in the same liquid phase (B). Here, “accumulated” means a partition coefficient of the homogeneous catalyst

P=[concentration of homogeneous catalyst in liquid phase (B)]/[concentration of homogeneous catalyst in liquid phase (A)]

of >1. The homogeneous catalyst is generally selected by means of a simple experiment in which the partition coefficient of the desired homogeneous catalyst is experimentally determined under the planned process conditions.

Liquid phase (A) here is the raffinate in process stage 2).

Owing to their good solubility in tertiary amines, complexes comprising an element of group 8, 9 or 10 of the Periodic Table and at least one phosphine group having at least one unbranched or branched, acyclic or cyclic aliphatic radical having from 1 to 12 carbon atoms, where individual carbon atoms can also be substituted by >P—, are preferably used as homogeneous catalysts in the process of the invention. Branched cyclic aliphatic radicals therefore also include radicals such as —CH₂—C₆H₁₁. As suitable radicals, mention may be made by way of example of methyl, ethyl, 1-propyl, 2-propyl, 1-butyl, 2-butyl, 1-(2-methyl)propyl, 2-(2-methyl)propyl, 1-pentyl, 1-hexyl, 1-heptyl, 1-octyl, 1-nonyl, 1-decyl, 1-undecyl, 1-dodecyl, cyclopentyl, cyclohexyl, cycloheptyl and cyclooctyl, methylcyclopentyl, methylcyclohexyl, 1-(2-methyl)pentyl, 1-(2-ethyl)hexyl, 1-(2-propyl)heptyl and norbornyl. The unbranched or branched, acyclic or cyclic aliphatic radical preferably comprises at least one carbon atom and preferably not more than 10 carbon atoms. In the case of an exclusively cyclic radical in the above sense, the number of carbon atoms is from 3 to 12 and preferably at least 4 and also preferably not more than 8. Preferred radicals are ethyl, 1-butyl, sec-butyl, 1-octyl and cyclohexyl.

The phosphine group can comprise one, two or three of the abovementioned unbranched or branched, acyclic or cyclic aliphatic radicals. These can be identical or different. The phosphine group preferably comprises three of the abovementioned unbranched or branched, acyclic or cyclic aliphatic radicals, with particular preference being given to all three radicals being identical. Preferred phosphines are P(n-CₙH₂ₙ₊₁)₃ where n is from 1 to 10, particularly preferably tri-n-butylphosphine, tri-n-octylphosphine and 1,2-bis(dicyclohexylphosphino)ethane.

As mentioned above, individual carbon atoms can also be substituted by >P— in the abovementioned unbranched or branched, acyclic or cyclic aliphatic radicals, Thus, polydentate, for example bidentate or tridentate, phosphine ligands are thus also encompassed. These preferably comprise the group >P—CH₂CH₂—P< or

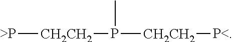

If the phosphine group comprises further radicals other than the abovementioned unbranched or branched, acyclic or cyclic aliphatic radicals, these generally correspond to those which are otherwise customarily used in phosphine ligands for complex catalysts. Examples which may be mentioned are phenyl, tolyl and xylyl.

The complex can comprise one or more, for example, two, three or four, of the abovementioned phosphine groups having at least one unbranched or branched, acyclic or cyclic aliphatic radical. The remaining ligands of the complex can have various natures. Illustrative examples which may be mentioned are hydride, fluoride, chloride, bromide, iodide, formate, acetate, propionate, carboxylate, acetylacetonate, carbonyl, DMSO, hydroxide, trialkylamine, alkoxide.

The homogeneous catalysts can be produced either directly in their active form or only under reaction conditions from conventional standard complexes such as [M(p-cymene)Cl₂]₂, [M(benzene)Cl₂]ₙ, [M(COD)(allyl)], [l MCl₃×H₂O], [M(acetylacetonate)₃], [M(DMSO)₄Cl₂] where M is an element of group 8, 9 or 10 of the Periodic Table with addition of the appropriate phosphine ligand(s).

Homogeneous catalysts which are preferred in the process of the invention are [Ru(PⁿBu₃)₄(H)₂], [Ru(Pⁿoctyl₃)₄(H)₂], [Ru(PⁿBu₃)₂(1,2-bis(dicyclohexylphosphino)-ethane)(H)₂], [Ru(Pⁿoctyl₃)₂(1,2-bis(dicyclohexylphosphino)ethane)(H)₂], [Ru(PEt₃)₄(H)₂]. These enable TOF (turnover frequency) values of greater than 1000 h⁻¹ to be achieved in the hydrogenation of carbon dioxide.

When homogeneous catalysts are used, the amount of the specified metal component in the metal-organic complex used is generally from 0.1 to 5000 ppm by weight, preferably from 1 to 800 ppm by weight and particularly preferably from 5 to 800 ppm by weight, in each case based on the total liquid reaction mixture in the hydrogenation reactor.

The partition coefficient of the homogeneous catalyst based on the amount of ruthenium in the amine phase and the product phase comprising the formic acid/amine adduct is in the range of P greater than 0.5, preferably greater than 1.0 and particularly preferably greater than 4, after the hydrogenation.

The tertiary amine to be used in the hydrogenation of carbon dioxide in the process of the invention has a boiling point which is at least 5° C. higher than that of formic acid. Here, as is customary, when the relative position of boiling points of compounds is indicated, the boiling points each have to be based on the same pressure. The tertiary amine is selected in this way and matched to the polar solvent in which the tertiary amine accumulates in the upper phase in the hydrogenation reactor. Here, “accumulates/accumulated” means a proportion by weight of >50% of the free, i.e. not bound in the form of the formic acid/amine adduct, tertiary amine in the upper phase based on the total amount of free, tertiary amine in the two liquid phases. The proportion by weight is preferably >90%. The tertiary amine is generally selected by means of a simple experiment in which the solubility of the desired tertiary amine in the two liquid phases is experimentally determined under the planned process conditions. The upper phase can additionally comprise amounts of the polar solvent and of a nonpolar inert solvent.

The tertiary amine to be used preferably has a boiling point which is at least 10° C. higher, particularly preferably at least 50° C. higher and very particularly preferably at least 100° C. higher, than that of formic acid. A restriction in terms of an upper limit for the boiling point is not necessary since a very low vapor pressure of the tertiary amine is in principle advantageous for the process of the invention. In general, the boiling point of the tertiary amine at a pressure of 1013 hPa abs, if necessary extrapolated from vacuum by known methods, is less than 500° C.

The tertiary amine which is preferably to be used in the process of the invention is an amine comprising at least 12 carbon atoms per molecule of the general formula (Ia)

NR¹R²R³ (Ia),

where the radicals R¹ to R³ are identical or different and are each, independently of one another, an unbranched or branched, acyclic or cyclic, aliphatic, araliphatic or aromatic radical having in each case from 1 to 16 carbon atoms, preferably from 1 to 12 carbon atoms, where individual carbon atoms can also, independently of one another, be substituted by a heterogroup selected from the group consisting of —O— and >N— and two or all three of the radicals can also be joined to one another to form a chain comprising at least four atoms.

As suitable amines, mention may be made by way of example of:
tri-n-butylamine, tri-n-pentylamine, tri-n-hexylamine, tri-n-heptylamine, tri-n-octylamine, tri-n-nonylamine, tri-n-decylamine, tri-n-undecylamine, tri-n-dodecylamine, tri-n-tridecylamine, tri-n-tetradecylamine, tri-n-pentadecylamine, tri-n-hexadecylamine, tri-(2-ethylhexyl)amine.
dimethyldecylamine, dimethyldodecylamine, dimethyltetradecylamine, ethyldi(2-propyl)amine (bp_{1013 hPa}=127° C.), dioctylmethylamine, dihexylmethylamine.
tricyclopentylamine, tricyclohexylamine, tricycloheptylamine, tricyclooctylamine and the derivatives thereof substituted by one or more methyl, ethyl, 1-propyl, 2-propyl, 1-butyl, 2-butyl or 2-methyl-2-propyl groups.
dimethylcyclohexylamine, methyldicyclohexylamine, diethylcyclohexylamine, ethyldicyclohexylamine, dimethylcyclopentylamine, methyldicyclopentylamine.
triphenylamine, methyldiphenylamine, ethyldiphenylamine, propyldiphenylamine, butyldiphenylamine, 2-ethylhexyldiphenylamine, dimethylphenylamine, diethylphenylamine, dipropylphenylamine, dibutylphenylamine, bis(2-ethylhexyl)phenylamine, tribenzylamine, methyldibenzylamine, ethyldibenzylamine and the derivatives thereof substituted by one or more methyl, ethyl, 1-propyl, 2-propyl, 1-butyl, 2-butyl or 2-methyl-2-propyl groups.
N—C₁-C₁₂-alkylpiperidines, N,N-di-C₁-C₁₂-alkylpiperazines, N—C₁-C₁₂-alkylpyrrolidines, N—C₁-C₁₂-alkylimidazoles and derivatives thereof substituted by one or more methyl, ethyl, 1-propyl, 2-propyl, 1-butyl, 2-butyl or 2-methyl-2-propyl groups.
1,8-diazabicyclo[5.4.0]undec-7-ene (“DBU”), 1,4-diazabicyclo[2.2.2]octane (“DABCO”), N-methyl-8-azabicyclo[3.2.1]octane (“tropane”), N-methyl-9-azabicyclo[3.3.1]nonane (“granatane”), 1-azabicyclo[2.2.2]octane (“quinuclidine”).

It is naturally also possible to use mixtures of any number of different tertiary amines in the process of the invention.

Particular preference is given to using an amine of the general formula (Ia) in which the radicals R¹ to R³ are selected independently from the group consisting of C₁-C₁₂-alkyl, C₅-C₈-cycloalkyl, benzyl and phenyl as tertiary amine in the process of the invention.

Particular preference is given to using a saturated amine, i.e. an amine comprising only single bonds, of the general formula (Ia) as tertiary amine in the process of the invention.

Very particular preference is given to using an amine of the general formula (la) in which the radicals R¹ to R³ are selected independently from the group consisting of C₅-C₈-alkyl, in particular tri-n-pentylamine, tri-n-hexylamine, tri-n-heptylamine, tri-n-octylamine, dimethylcyclohexylamine, methyldicyclohexylamine, dioctylmethylamine and dimethyldecylamine, as tertiary amine in the process of the invention.

In particular, an amine of the general formula (la) in which the radicals R¹ to R³ are selected independently from among C₅- and C₆-alkyl is used as tertiary amine.

In the process of the invention, the tertiary amine is preferably present in liquid form in all process stages. However, this is not an absolute requirement. It would also be sufficient for the tertiary amine to be dissolved in suitable solvents. Suitable solvents are in principle those which are chemically inert in respect of the hydrogenation of carbon dioxide and the thermal dissociation of the adduct and those in which the tertiary amine and, if a homogeneous catalyst is used, also the latter dissolve(s) readily while polar solvent and the formic acid/amine adducts are sparingly soluble. Possibilities are therefore in principle chemically inert, nonpolar solvents such as aliphatic, aromatic or araliphatic hydrocarbons, for example octane and higher alkanes, toluene, xylenes.

The polar solvent to be used in the hydrogenation of carbon dioxide in the process of the invention has a boiling point which is at least 5° C. lower than the temperature required for dissociation of the formic acid/amine adducts at the same pressure. The polar solvent should be selected or matched to the tertiary amine so that the lower phase is enriched in the polar solvent. Here, “enriched” means a proportion by weight of >50% of the polar solvent in the lower phase based on the total amount of polar solvent in the two liquid phases. The proportion by weight is preferably >70%. The polar solvent is generally selected by means of a simple experiment in which the solubility of the desired polar solvent in the two liquid phases is experimentally determined under the planned process conditions.

The polar solvent can be a pure polar solvent or a mixture of various polar solvents as long as the abovementioned conditions in respect of boiling point and phase behavior which the solvent has to meet are complied with.

The polar solvent to be used preferably has a boiling point which is at least 10° C. lower, particularly preferably less than 50° C. lower, than the temperature required for dissociation of the formic acid/amine adducts at the same pressure. In the case of solvent mixtures, the boiling points of the solvent mixture used or of an azeotrope or heteroazeotrope are at least 10° C. lower, particularly preferably at least 50° C. lower, than the temperature required for dissociation of the formic acid/amine adducts at the same pressure.

Classes of substances which are suitable as polar solvents are preferably alcohols and the formic esters thereof and water. The alcohols have a boiling point which is at least 10° C. lower, particularly preferably at least 50° C. lower, than the temperature required for dissociation of the formic acid/amine adducts at the same pressure so as to keep esterification of the alcohols by formic acid as low as possible.

Suitable alcohols are alcohols in the case of which the trialkylammonium formates preferably dissolve in a mixture of the alcohol with water and this product phase has a miscibility gap with the free trialkylamine. As suitable alcohols, mention may be made by way of example of methanol, ethanol, 2-methoxyethanol, 1-propanol, 2-propanol, 1-butanol, 2-butanol, 2-methyl-1-propanol. It is also possible to use mixtures of one or more alcohols and water as polar solvent. In one embodiment of the invention, a mixture of one or more monoalcohols selected from among methanol, ethanol, propanols and butanols with water is used as polar solvent. In a further embodiment, a mixture of methanol and/or ethanol with water is used as polar solvent. The ratio of alcohol to water should be selected so that the mixture together with the trialkylammonium formate and the trialkylamine forms a two-phase mixture in which the major part of the trialkylammonium formate, the water and the polar solvent is present in the lower phase, which is generally determined by means of a simple experiment in which the solubility of the desired polar solvent mixture in the two liquid phases is experimentally determined under the planned process conditions.

The molar ratio of the polar solvent or solvent mixture to be used in the process of the invention to the tertiary amine used is generally from 0.5 to 30 and preferably from 1 to 20.

In an embodiment of the invention, water is added to the output (3) from the hydrogenation reactor (I) before the output (3) is fed to the extraction unit (II).

The invention therefore also provides a process for preparing formic acid by reacting carbon dioxide (1) with hydrogen (2) in a hydrogenation reactor (I) in the presence of
a catalyst comprising an element of group 8, 9 or 10 of the Periodic Table,
a tertiary amine comprising at least 12 carbon atoms per molecule and
a polar solvent comprising one or more monoalcohols selected from among methanol, ethanol, propanols and butanols,

to form formic acid/amine adducts as intermediates which are subsequently thermally dissociated,

where a tertiary amine having a boiling point which is at least 5° C. higher than that of formic acid is used and

a reaction mixture comprising the polar solvent, the formic acid/amine adducts, the tertiary amine and the catalyst is formed in the reaction in the hydrogenation reactor (I) and is discharged from the reactor as output (3),

wherein

the output (3) from the hydrogenation reactor (I) is, after addition of water, fed directly to an extraction unit (II) and the work-up of the output (3) comprises the following process steps:
1) extraction of the catalyst from the output (3) from the hydrogenation reactor (I) in the extraction unit (II), where the same tertiary amine which was used in the hydrogenation is used as extractant, to give an extract (4) which comprises the major part of the tertiary amine and the catalyst and is recycled to the hydrogenation reactor (I) and also a raffinate (5) which comprises the major part of the polar solvent and the formic acid/amine adducts and is passed on to a distillation unit (III) in which
2) a distillation is carried out to give an overhead stream (6) which comprises predominantly the polar solvent and is recycled to the hydrogenation reactor (I) and also a bottom stream (7) which comprises predominantly the formic acid/amine adducts and the tertiary amine and is according to process step
3) fed to a phase separation vessel (IV) in which
a) a phase separation is carried out to give an upper phase which comprises predominantly the tertiary amine and is recycled as stream (11) to the extraction unit (II) as extractant for the catalyst and a lower phase which comprises predominantly the formic acid/amine adducts and is fed as stream (8) to a thermal dissociation unit (V) in which
b) a thermal dissociation is carried out to give a stream (9) which comprises the tertiary amine and is recirculated to the phase separation vessel (IV) and a stream (10) comprising the formic acid or according to process step
4) is fed to a thermal dissociation unit (V) in which
a) a thermal dissociation is carried out to give a stream (10) comprising the formic acid and a stream (9) which comprises the tertiary amine and the formic acid/amine adducts and is fed to a phase separation vessel (IV) in which
b) a phase separation is carried out to give an upper phase which comprises predominantly the tertiary amine and is recycled as stream (11) to the extraction unit (II) as extractant for the catalyst and a lower phase which comprises predominantly the formic acid/amine adducts and is fed as stream (8) to a thermal dissociation unit (V).

The addition of water after hydrogenation has been carried out has the effect that the partition coefficients of the catalyst are improved by the addition of water in favor of accumulation of the catalyst in the amine phase and that in this way efficient recirculation of the catalyst is made possible without the hydrogenation activity thereof being reduced. The hydrogenation output can, depending on the polar solvent used and the concentration of formic acid-amine adducts, consist of one or two phases, with the formic acid-amine adducts then accumulating in the product phase (raffinate) as result of the addition of water. Water is preferably added in such an amount that a water content in the raffinate (5) of from 0.1 to 50% by weight, based on the total weight of the raffinate (5) from the extraction unit (II), is obtained, particularly preferably from 2 to 30% by weight, based on the total weight of the raffinate (5) from the extraction unit (H). The water to be introduced can originate from the distillation unit (III) in which the major part of the polar solvent and also the water are separated off from the raffinate (5) and/or can also be water which is freshly introduced into the process. The water can, when the output (3) is depressurized, be added either after depressurization of the output from the hydrogenation reactor to atmospheric pressure or else before depressurization of the output.

In a further embodiment of the invention, no water is added to the output (3) from the hydrogenation reactor (I). The invention therefore also provides a process for preparing formic acid by reacting carbon dioxide (1) with hydrogen (2) in a hydrogenation reactor (I) in the presence of
a catalyst comprising an element of group 8, 9 or 10 of the Periodic Table,
a tertiary amine comprising at least 12 carbon atoms per molecule and
a polar solvent comprising one or more monoalcohols selected from among methanol, ethanol, propanols, butanols and water,

to form formic acid/amine adducts as intermediates which are subsequently thermally dissociated,

where a tertiary amine having a boiling point which is at least 5° C. higher than that of formic acid is used and

a reaction mixture comprising the polar solvent, the formic acid/amine adducts, the tertiary amine and the catalyst is formed in the reaction in the hydrogenation reactor (I) and is discharged from the reactor as output (3),

wherein

the output (3) from the hydrogenation reactor (I) is fed directly to an extraction unit (II) and the work-up of the output (3) comprises the following process steps:
1) extraction of the catalyst from the output (3) from the hydrogenation reactor (I) in the extraction unit (II), where the same tertiary amine which was used in the hydrogenation is used as extractant, to give an extract (4) which comprises the major part of the tertiary amine and the catalyst and is recycled to the hydrogenation reactor (I) and also a raffinate (5) which comprises the major part of the polar solvent and the formic acid/amine adducts and is passed on to a distillation unit (III) in which
2) a distillation is carried out to give an overhead stream (6) which comprises predominantly the polar solvent and is recycled to the hydrogenation reactor (I) and also a bottom stream (7) which comprises predominantly the formic acid/amine adducts and the tertiary amine and is according to process step
3) fed to a phase separation vessel (IV) in which
a) a phase separation is carried out to give an upper phase which comprises predominantly the tertiary amine and is recycled as stream (11) to the extraction unit (II) as extractant for the catalyst and a lower phase which comprises predominantly the formic acid/amine adducts and is fed as stream (8) to a thermal dissociation unit (V) in which
b) a thermal dissociation is carried out to give a stream (9) which comprises the tertiary amine and is recirculated to the phase separation vessel (IV) and a stream (10) comprising the formic acid or according to process step
4) is fed to a thermal dissociation unit (V) in which
a) a thermal dissociation is carried out to give a stream (10) comprising the formic acid and a stream (9) which comprises the tertiary amine and the formic acid/amine adducts and is fed to a phase separation vessel (IV) in which
b) a phase separation is carried out to give an upper phase which comprises predominantly the tertiary amine and is recycled as stream (11) to the extraction unit (II) as extractant for the catalyst and a lower phase which comprises predominantly the formic acid/amine adducts and is fed as stream (8) to a thermal dissociation unit (V).

Here, the ratios of monoalcohol(s), tertiary amine and water in the hydrogenation reactor (I) are preferably selected so that a water content in the raffinate (5) of from 0.1 to 50% by weight, based on the total weight of the raffinate (5) from the extraction unit (II), is obtained, particularly preferably from 2 to 30% by weight, based on the total weight of the raffinate (5) from the extraction unit II.

The extract (4) obtained in the extraction unit (II) comprises the major part of the tertiary amine and the catalyst. The raffinate (5) obtained in the extraction unit (II) comprises the major part of the polar solvent and the formic acid/amine adducts.

“Major part” in relation to the extract (4) means that the extract (4) has a higher concentration of tertiary amine and catalyst than the raffinate (5). Here, tertiary amine is the amine which is present in free form and is not bound to formic acid in the form of the formic acid/amine adduct.

“Major part” in relation to the raffinate (5) means that the raffinate (5) has a higher concentration of polar solvent and the formic acid/amine adducts than the extract (4).

In one embodiment of the process of the invention, a substream of the bottom stream (7) obtained in the distillation unit (III) is fed to the phase separation vessel (IV) and the remainder of the bottom stream (7) is fed to the thermal dissociation unit (V). A substream of the bottom stream (7) is worked up according to process step 3) and the remainder of the bottom stream is worked up according to process step 4).

The carbon dioxide to be used in the hydrogenation of carbon dioxide can be used in solid, liquid or gaseous form. It is also possible to use industrially available gas mixtures comprising carbon dioxide as long as these are largely free of carbon monoxide, i.e. have a proportion by volume of <1% of CO. The hydrogen to be used in the hydrogenation of carbon dioxide is generally gaseous. Carbon dioxide and hydrogen can also comprise inert gases such as nitrogen or noble gases. However, the content of these is advantageously below 10 mol % based on the total amount of carbon dioxide and hydrogen in the hydrogenation reactor. Although larger amounts may likewise still be tolerable, they generally require use of a higher pressure in the reactor, as a result of which further compression energy is required and the outlay in terms of apparatus increases.

The hydrogenation of carbon dioxide is carried out in the liquid phase, preferably at a temperature of from 20 to 200° C. and a total pressure of from 0.2 to 30 MPa abs. The temperature is preferably at least 30° C. and particularly preferably at least 40° C. and preferably not more than 150° C., particularly preferably not more than 120° C. and very particularly preferably not more than 80° C. The total pressure is preferably at least 1 MPa abs and particularly preferably at least 3 MPa abs and preferably not more than 15 MPa abs.

The partial pressure of the carbon dioxide is generally at least 0.5 MPa and preferably at least 1 MPa and generally not more than 8 MPa. The partial pressure of the hydrogen is generally at least 0.5 MPa and preferably at least 1 MPa and generally not more than 25 MPa and preferably not more than 10 MPa.

The molar ratio of hydrogen to carbon dioxide in the feed to the hydrogenation reactor is preferably from 0.1 to 10 and particularly preferably from 0.2 to 5, in particular from 0.5 to 3.

The molar ratio of carbon dioxide to tertiary amine in the feed to the hydrogenation reactor is generally from 0.1 to 10 and preferably from 0.2 to 5, in particular from 0.5 to 3.

As hydrogenation reactors, it is in principle possible to use all reactors which are basically suitable for gas/liquid reactions at the given temperature and the given pressure. Suitable standard reactors for gas-liquid and for liquid-liquid reaction systems are indicated, for example, in K. D. Henkel, “Reactor Types and Their Industrial Applications”, in Ullmann's Encyclopedia of Industrial Chemistry, 2005, Wiley-VCH Verlag GmbH & Co. KGaA, DOI: 10.1002/14356007.b04_{—}087, chapter 3.3 “Reactors for gas-liquid reactions”. Examples which may be mentioned are stirred tank reactors, tube reactors and bubble column reactors.

The hydrogenation of carbon dioxide can be carried out batchwise or continuously in the process of the invention. In batch operation, the reactor is charged with the desired liquid and optionally solid starting materials and auxiliaries and the reactor is subsequently pressurized with carbon dioxide and hydrogen to the desired pressure at the desired temperature. After the reaction is complete, the reactor is generally depressurized and the two liquid phases formed are separated from one another. In continuous operation, the starting materials and auxiliaries, including the carbon dioxide and hydrogen, are introduced continuously. Correspondingly, the liquid phase is continuously discharged from the reactor so that the liquid level in the reactor stays the same on average. Preference is given to the continuous hydrogenation of carbon dioxide.

The average residence time in the hydrogenation reactor is generally from 5 minutes to 5 hours.

The formic acid/amine adducts formed in the hydrogenation of carbon dioxide in the presence of the catalyst to be used, the polar solvent and the tertiary amine generally have the general formula (IIa)

NR¹R²R³·xᵢHCOOH (IIa),

where the radicals R¹ to R³ correspond to the radicals described for the tertiary amine (Ia) and x₁ is from 0.4 to 5, preferably from 0.7 to 1.6. The respective average composition of the amine-formic acid ratio in the product phases in the respective process steps, i.e. the factor xᵢ, can be determined, for example, by determining the formic acid content by titration with an alcoholic KOH solution against phenolphthalein and the amine content by gas chromatography. The composition of the formic acid/amine adducts, i.e. the factor xᵢ, can change during the various process steps. Thus, for example, adducts having a relatively high formic acid content with x₂>x₁ and x₂ from 1 to 4 are generally formed after removal of the polar solvent, with the excess, free amine being able to form a second phase.

The output (3) from the hydrogenation reactor (I), which comprises the polar solvent, the formic acid/amine adducts, the tertiary amine and the catalyst and to which water has optionally been added, is extracted with streams of free tertiary amine originating from the respective phase separation vessels and recirculated to the hydrogenation reactor. This is done to separate off the catalyst. Without this extraction, hydrogenation catalyst could get into the apparatus for thermal dissociation of the adduct of tertiary amine and formic acid and there catalyze the decomposition of formic acid and thus reduce the yield of formic acid. Residual amounts of hydrogen and carbon dioxide are disposed of as offgas.

The extraction of the catalyst from the output from the hydrogenation reactor can, for example, be carried out after depressurization, for example to about or close to atmospheric pressure, and cooling of the output (3), for example to about or close to ambient temperature.

Here, the extract (4) obtained in the extraction, which comprises the tertiary amine and the catalyst, should be brought separately to the reaction pressure before recirculation to the hydrogenation reactor (I). One or more suitable compressors designed for the pressure difference to be overcome or a pump are provided for the gas and liquid phases to be recirculated.

In the context of the present invention, it has surprisingly been found that a raffinate (5) enriched in the formic acid/amine adducts and the polar solvent and an extract (4) enriched in the tertiary amine and, if a homogeneous catalyst is used, also in this can be obtained in the extraction, even at a significantly elevated pressure. For this reason, the solvents can be selected in the process of the invention so that separation of the phase enriched in the formic acid/amine adducts and the polar solvent from the other phase enriched in the tertiary amine and also recirculation of the phase enriched in tertiary amine to the hydrogenation reactor at a pressure of from 0.1 to 30 MPa abs can take place. In accordance with the total pressure in the hydrogenation reactor, the pressure is preferably not more than 15 MPa abs. It is therefore even possible to separate the two liquid phases from one another without prior depressurization and to recirculate the extract (4) from the extraction to the hydrogenation reactor without an appreciable increase in pressure. In this case, and also in the case of only slight depressurization, it is possible to dispense with recirculation of any gas phase entirely.

The process of the invention can, in one embodiment, therefore be carried out with the pressure and the temperature in the hydrogenation reactor and in the extraction unit (II) being the same or approximately the same, where, for the present purposes, “approximately the same” means a pressure difference of up to +/−5 bar or a temperature difference of up to +/−5° C.

It has also surprisingly been found that in the present system the two liquid phases can be separated very well from one another even at an elevated temperature corresponding to the reaction temperature. It is therefore also not necessary to carry out any cooling for the phase separation and subsequent heating of the upper phase to be recirculated, which likewise saves energy.

The extraction is preferably carried out at temperatures in the range from 20 to 100° C., particularly preferably from 40 to 80° C.

The extraction of the hydrogenation catalyst can be carried out in any suitable apparatus known to those skilled in the art, preferably in countercurrent extraction columns, cascades of mixer-settlers or combinations of mixer-settlers with columns.

The raffinate (5) from the extraction unit (II), which comprises the major part of the polar solvent and the formic acid/amine adducts, is fed to the distillation unit (III) in order to separate the polar solvent or solvent mixture from the formic acid/amine adduct. The phase comprising tertiary amine and hydrogenation catalyst from the extraction unit (II) is recirculated to the hydrogenation reactor.

Amounts of individual components of the polar solvent in the liquid phase to be extracted are sometimes dissolved in addition to the catalyst in the extractant, viz, the amine stream, This is not a disadvantage for the process since the amount of solvent already extracted does not have to be fed to the solvent removal and may thus save vaporization energy and apparatus costs.

It can be advantageous to integrate an apparatus for adsorbing traces of hydrogenation catalyst into the plant for carrying out the process between the extraction unit (II) and the distillation unit (III). Numerous adsorbents are suitable for this absorption. Examples are polyacrylic acid and salts thereof, sulfonated polystyrenes and salts thereof, activated carbons, montmorillonites, bentonites, silica gels and zeolites.

In the extraction of the catalyst from the output (3) in the extraction unit (II), an extract (4) comprising the tertiary amine and the catalyst and a raffinate (5) comprising the polar solvent and the formic acid/amine adducts are obtained. The raffinate (5) is enriched in the formic acid/amine adducts and the polar solvent. With regard to the formic acid/amine adducts, “enriched” means a partition coefficient of the formic acid/amine adducts

P=[concentration of formic acid/amine adduct (II) in liquid phase (A)]/[concentration of formic acid/amine adduct (II) in liquid phase (B)]

of >1. The partition coefficient is preferably ≧2 and particularly preferably ≧5. The extract (4) is enriched in the tertiary amine. When a homogeneous catalyst is used, this likewise accumulates in the raffinate. The extract (4) is recycled to the hydrogenation reactor.

The liquid phases (A) and (B) have the meaning defined above.

Recirculation of a gas phase comprising unreacted carbon dioxide and/or unreacted hydrogen to the hydrogenation reactor may also be advantageous. This may be desirable, for example, for discharging undesirable by-products or impurities, part of the upper phase and/or part of the liquid or gaseous phases comprising carbon dioxide or carbon dioxide and hydrogen from the process.

The major part of the polar solvent of the raffinate (5) is separated off thermally from the formic acid/amine adducts in a distillation unit (III), with the polar solvent which has been removed by distillation being recirculated to the hydrogenation reactor (I). The pure formic acid/amine adducts and also free tertiary amine are obtained at the bottom of the distillation unit (III) since formic acid/amine adducts having a relatively low amine content are formed on removal of the polar solvent, as a result of which a two-phase bottoms mixture comprising an amine phase and a formic acid/amine adduct phase is formed.

The thermal separation of the polar solvent or solvent mixture, see above, is preferably carried out at a temperature at the bottom at which, at the given pressure, no free formic acid is formed from the formic acid/amine adduct having the higher (×1) or lower (×2) amine content. In general, the temperature at the bottom of the thermal separation unit is at least 20° C., preferably at least 50° C. and particularly preferably at least 70° C. and generally not more than 210° C., preferably not more than 190° C. The pressure is generally at least 1 hPa abs, preferably at least 50 hPa abs and particularly preferably at least 100 hPa abs and generally not more than 1 MPa abs and preferably 0.1 MPa abs.

The thermal removal of the polar solvent or solvent mixture is carried out either in an evaporator or in a distillation unit comprising an evaporator and column, filled with ordered packing, random packing elements and/or trays. The solvent can be condensed after the thermal separation, and the enthalpy of condensation liberated can in turn be utilized, for example, to preheat the solvent with amine/formic acid adduct mixture coming from the extraction to evaporation temperature.

As an alternative, only parts of the solvent mixture can be separated off. This applies particularly to solvent components which can be separated off via a side stream in the later formic acid distillation.

The formic acid/amine adducts which are obtained after the thermal removal of the polar solvent or solvent mixture or parts of the solvent in the distillation unit (III) are then, as bottom stream (7), thermally dissociated into free formic acid and free tertiary amine in a thermal dissociation unit (V), for example a distillation unit, with the free formic acid formed being distillatively removed and the free tertiary amine comprised in the bottoms from the distillation unit being recirculated to the hydrogenation reactor (I). Here, the free amine obtained as second phase in the thermal removal of the polar solvent in the distillation unit (III) can be separated off beforehand in a phase separation vessel, together with the bottom product from the thermal dissociation unit (V) for isolation of formic acid in a common phase separation vessel or fed directly as two-phase mixture to the dissociation unit (V) (see general embodiments). The liberated formic acid can be taken off, for example, (i) at the top, (ii) at the top and as side offtake stream or (iii) only as side offtake stream. If formic acid is taken off at the top, a formic acid purity of up to 99.99% by weight is possible. When formic acid is taken off as side offtake stream, aqueous formic acid is obtained, with the mixture comprising about 85% by weight of formic acid being of particular importance in industrial practice. Depending on the water content of the feed to the distillation unit, the formic acid is taken off to an increased extent as overhead product or as side product. If necessary, it is even possible to take off formic acid only as side product, in which case the required amount of water may even not be able to be explicitly added. The thermal dissociation of the formic acid/amine adduct is generally carried out at the process parameters in respect of pressure, temperature and configuration of the apparatus which are known from the prior art. Reference may thus be made, for example, to the descriptions in EP 0 181 078 A or WO 2006/021411. The distillation unit to be used generally comprises a distillation column which generally comprises random packing elements, ordered packings and/or trays.

In general, the temperature at the bottom of the distillation column (dissociation unit (V)) is generally at least 130° C., preferably at least 140° C. and particularly preferably at least 150° C., and generally not more than 210° C., preferably not more than 190° C. and particularly preferably not more than 185° C. The pressure is generally at least 1 hPa abs, preferably at least 50 hPa abs and particularly preferably at least 100 hPa abs, and generally not more than 500 hPa abs, preferably not more than 300 hPa abs and particularly preferably not more than 250 hPa abs.

A water-comprising stream of formic acid may optionally also be taken off as side product.

The process of the invention has a number of advantages over the integrated processes which have previously been described in EP 181 078 B1 and EP 357 243 B1: the same tertiary amine is used for binding the formic acid in the hydrogenation and the thermal dissociation of the formic acid/amine adducts. This amine, which is obtained in free form in the thermal dissociation, is then used for extraction of the catalyst from the product phase in order to recirculate the catalyst together with the amine to the reaction vessel. It has a higher stability than the N-alkylimidazoles previously described. Losses of noble metals virtually do not occur. The catalyst is prevented from getting into the thermal dissociation unit and there catalyzing the decomposition of formic acid. It is a great advantage that it can be separated off in its active form and recirculated. High formic acid yields and a high product purity are achieved. The extraction replaces two distillation systems. As a result, energy costs and capital costs are reduced.

The process of the invention makes it possible to isolate concentrated formic acid in high yield and high purity by hydrogenation of carbon dioxide. It has, in particular, a particularly simple mode of operation which involves, compared to the prior art, a simpler process concept, simpler process stages, a smaller number of process stages and also simpler apparatuses. Thus, for example, an appropriate choice of the tertiary amine and of the polar solvent enables, in the case of the use of a homogeneous catalyst, the latter to be separated off by extraction of formic acid/amine adducts and recirculated without further work-up steps to the hydrogenation reactor. The extraction can also be carried out under superatmospheric pressure. Owing to the prompt separation of the catalyst from the formic acid/amine adducts formed, a backreaction with decomposition into carbon dioxide and hydrogen is suppressed.

Furthermore, no complicated, separate base replacement is necessary in the process of the invention, so that the formic acid/amine adducts formed in the hydrogenation reactor can be used directly for thermal dissociation. The use of a low-boiling polar solvent makes it possible for this solvent to be separated off thermally under mild conditions in a stage preceding the thermal dissociation of formic acid, as a result of which esterification of alcohols used and decomposition of the formic acid are minimized, a lower energy consumption is necessary and a higher purity of the formic acid can be achieved. As a result of the simpler process concept, the production plant required for carrying out the process of the invention is more compact in terms of a lower space requirement and the use of fewer apparatuses compared to the prior art. It has a lower capital outlay and a lower energy consumption.

The invention is illustrated below with the aid of examples and a drawing.

### EXAMPLE A1-4

### Hydrogenation, Catalyst Extraction and Reuse of the Catalyst

A 100 ml or 250 ml autoclave made of Hastelloy C and equipped with a blade stirrer or magnetic stirrer was charged under inert conditions with the trialkylamine, polar solvent and the catalyst. The autoclave was subsequently closed and CO₂ was injected at room temperature. H₂ was subsequently injected and the reactor was heated while stirring (700-1000 rpm). After the reaction time, the autoclave was cooled and the reaction mixture was depressurized. After the reaction, trialkylamine and optionally water were added to the reaction output to effect extraction and the mixture was stirred at room temperature for 10 minutes. A two-phase product mixture was obtained, with the upper phase being enriched in the tertiary amine and the homogeneous catalyst and the lower phase being enriched in the polar solvent and the formic acid/amine adduct formed. The phases were subsequently separated and the formic acid content of the lower phase was determined. The upper phase comprising ruthenium catalyst and the free amine was then reused as before for the CO₂ hydrogenation with the same solvent under the same reaction conditions. After the reaction was complete, fresh trialkylamine was added in order to extract the catalyst. The product phase was subsequently separated off and the formic acid content and also the ruthenium content of the two phases were determined. The total content of formic acid in the formic acid/amine adduct was determined by potentiometric titration with 0.1 N KOH in MeOH using a “Mettler Toledo DL50” titrator. The ruthenium content was determined by AAS. The parameters and results of the individual experiments are shown in Tables 1.1 to 1.2.

Examples A-1 to A-4 show that the active catalyst can be separated off from the product phase by extraction and reused for the hydrogenation. Relatively high degrees of enrichment for the ruthenium can be achieved by simple connection of a plurality of extraction steps in series, e.g. in a cascade of mixer-settlers or in a countercurrent extraction, using the same amount of amine.

TABLE 1.1

| | Example A-1a (first hydrogenation | Example A-1b (reuse of the | Example A-2a (first | Example A-2b (reuse of the |
|---|---|---|---|---|
| | and extraction) | catalyst and extraction) | hydrogenation) | catalyst and extraction) |
| Autoclave | 250 ml | 250 ml | 100 ml | 100 ml |
| Tertiary amine | 65.0 g of trihexylamine | Upper phase from A-1a | 20.0 g of trihexylamine | 15.0 g of upper phase from |
| | | | | A-2a |
| Polar solvent | 25.0 g of methanol | 25.0 g of methanol | 10.0 g of methanol | 10.0 g of methanol |
| (introduced) | 2.0 g of water | 2.0 g of water | 1.0 g of water | 1.0 g of water |
| Catalyst | 0.32 g of [Ru(PnOctyl₃)₄(H)₂], | Upper phase from A-1a | 0.16 g of [Ru(PnOctyl₃)₄(H)₂], | 15.0 g of upper phase from |
| | 0.08 g of 1,2- | | 0.04 g of 1,2- | A-2 |
| | bis(dicyclohexylphosphino)ethane | | bis(dicyclohexylphosphino)ethane | |
| Injection of CO₂ | To 3.1 MPa abs | To 3.0 MPa abs | To 2.2 MPa abs | To 2.4 MPa abs. |
| Injection of H₂ | To 12.0 MPa abs | To 12.0 MPa | To 8.0 MPa abs | To 8.0 MPa abs |
| Heating | 70° C. | 70° C. | 70° C. | 70° C. |
| Reaction time | 1 hour | 1 hour | 1 hour | 1 hour |
| Amine addition to | 65.0 g of trihexylamine | 65.0 g of trihexylamine | 15.0 g of trihexylamine | 15.1 g of trihexylamine |
| the extraction | | | | |
| after the reaction | | | | |
| Water addition | — | 4.0 g | — | — |
| after the reaction | | | | |
| Upper phase | 77.3 g | 118.0 g | 19.6 g | 25.8 g |
| Lower phase | 80.9 g | 50.5 g | 26.0 g | 17.6 g |
| | 8.6% of formic acid | 6.4% of formic acid | 7.73% of formic acid | 5.3% of formic acid |
| c_{Ru} in | — | 210 ppm | 290 ppm | 170 ppm |
| trialkylamine phase | | | | |
| after extraction | | | | |
| c_{Ru} in lower | — | 3 ppm | 75 ppm | 18 ppm |
| phase after | | | | |
| extraction | | | | |

TABLE 1.2

| | Example A-3a (first hydrogenation | Example A-3b (reuse of | Example A-4a (first | Example A-4b (reuse of the |
|---|---|---|---|---|
| | and extraction) | the catalyst and extraction) | hydrogenation) | catalyst and extraction) |
| Autoclave | 250 ml | 250 ml | 250 ml | 250 ml |
| Tertiary amine | 65.0 g of trihexylamine | Upper phase from A-3a | 65.0 g of trihexylamine | Upper phase from A-4a |
| Polar solvent | 25.0 g of methanol | 25.0 g of methanol | 25.0 g of methanol | 25.0 g of methanol |
| (introduced) | | | 3.0 g of water | |
| Catalyst | 0.16 g of [Ru(PnOctyl₃)₄(H)₂], | Upper phase from A-3a | 0.16 g of [Ru(PnOctyl₃)₄(H)₂], | Upper phase from A-4a |
| | 0.04 g of 1,2- | | 0.04 g of 1,2- | |
| | bis(dicyclohexylphosphino)ethane | | bis(dicyclohexylphosphino)ethane | |
| Injection of CO₂ | To 2.4 MPa abs | To 2.3 MPa abs | To 2.7 MPa abs | To 2.2 MPa abs |
| Injection of H₂ | To 12.0 MPa abs | To 12.0 MPa | To 12.0 MPa abs | To 12.0 MPa |
| Heating | 70° C. | 70° C. | 70° C. | 70° C. |
| Reaction time | 1 hour | 1 hour | 1 hour | 1 hour |
| Amine addition to | 66.0 g of trihexylamine | — | 66.6 g of trihexylamine | — |
| the extraction | | | | |
| after the reaction | | | | |
| Water addition | 4.1 g | 4.0 g | — | — |
| after the reaction | | | | |
| Upper phase | 97.7 g | 67.3 g | 91.5 g | 67.0 g |
| Lower phase | 61.7 g | 54.6 g | 69.1 g | 50.0 g |
| | 8.3% of formic acid | 6.0% of formic acid | 8.6% of formic acid | 5.0% of formic acid |
| c_{Ru} in | 75 ppm | 95 ppm | 80 ppm | 100 ppm |
| trialkylamine phase | | | | |
| after extraction | | | | |
| c_{Ru} in lower | 27 ppm | 4 ppm | 25 ppm | 4 ppm |
| phase after | | | | |
| extraction | | | | |

### EXAMPLES B1 AND B2

### Thermal Removal of the Polar Solvent from the Trialkylamine/Solvent/Formic Acid Mixtures and Dissociation of the Formic Acid-Amine Adducts; Process Steps 3 and 4

Alcohol and water are distilled off from the product phase (comprises the formic acid/amine adduct) under reduced pressure by means of a rotary evaporator. A two-phase mixture (trialkylamine phase and formic acid/amine adduct phase) is formed as bottoms and the two phases are separated. The composition of the distillate (comprising the major part of the methanol and of the water), the upper phase (comprising the free trialkylamine) and the lower phase (comprising the formic acid-amine adduct) was determined by gas chromatography and by potentiometric titration of the formic acid against 0.1 N KOH in MeOH using a “Mettler Toledo DL50” titrator. The formic acid is then thermally split off from the trialkylamine in the lower phase from the first step in a vacuum distillation apparatus using a 10 cm Vigreux column. Complete removal of the formic acid leaves single-phase bottoms comprising the pure trialkylamine which can be used for extraction of the catalyst and recirculation to the hydrogenation. The formic acid and residual water are present in the distillate. The composition of the bottoms and of the distillate was determined by gas chromatography and by potentiometric titration of the formic acid against 0.1 N KOH in MeOH using a “Mettler Toledo DL50” titrator. The parameters and results of the individual experiments are shown in Table 1.3.

Examples B1 and B2 show that various polar solvents can be separated off under mild conditions from the product phase in the process of the invention, forming a lower phase which is relatively rich in formic acid and an upper phase comprising predominantly tertiary amine. The formic acid can then be split off from the trialkylamine in this lower phase which is relatively rich in formic acid at elevated temperatures, giving the free trialkylamine. The formic acid obtained in this way still comprises some water but this can be separated off from the formic acid by means of a column having a relatively high separation power. The trialkylamine obtained both in the removal of the solvent and also in the thermal dissociation can be used for removing the catalyst from the product stream in step 2.

TABLE 1.3

| | Example B-1a | Example B-1b | Example B-2a | Example B-2b |
|---|---|---|---|---|
| | (removal of the polar | (dissociation of the formic | (removal of the polar | (dissociation of the formic acid- |
| | solvent) | acid-amine adduct) | solvent) | amine adduct) |
| Feed mixture | 199.8 g | Lower phase from B1-a | 199.8 g | Lower phase from B2-a |
| (% by weight) | 8.9% of formic acid | | 7.8% of formic acid | |
| | 28.4% of methanol | | 33.0% of methanol | |
| | 5.6% of water | | 15.1% of water | |
| | 57.1% of trihexylamine | | 44.0% of trihexylamine | |
| Formic acid:amine in | 1:1.1 | 1:0.64 | 1:1 | 1:0.89 |
| feed mixture | | | | |
| Pressure | 200 mbar | 90 mbar | 200 mbar | 90 mbar |
| Temperature | 120° C. | 153° C. | 120° C. | 153° C. |
| Lower phase in the | 79.8 g | 63.6 g | 69.4 g | 55.5 g |
| bottoms after distillation | 22.1% of formic acid | 100% of trihexylamine | 14.9% of formic acid | 99.7% of trihexylamine |
| (% by weight) | 1.5% of water | | 6.9% of water | 0.3% of water |
| | 76.4% of trihexylamine | | 78.2% of trihexylamine | |
| Upper phase in the | 50.5 g | Single-phase | 32.7 g | Single-phase |
| bottoms after distillation | 100% of trihexylamine | | 99.7% of trihexylamine | |
| | | | 0.3% of water | |
| Distillate | 66.6 g | 14.9 g | 93.1 g | 12.9 g |
| | 0.3% of formic acid | 92.1% of formic acid | 70.1% of methanol | 85.0% of formic acid |
| | 81.2% of methanol | 7.9% of water | 29.9% of water | 15% of water |
| | 18.5% of water | | | |

In the drawings:

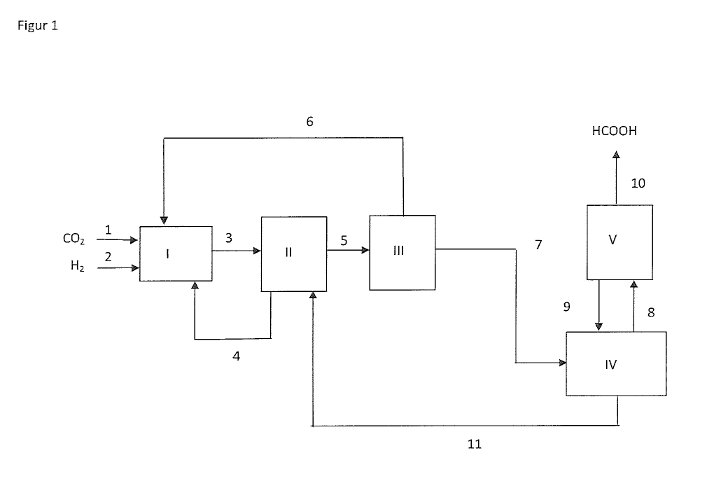

FIG. 1 shows a block diagram of a preferred embodiment of the process of the invention,

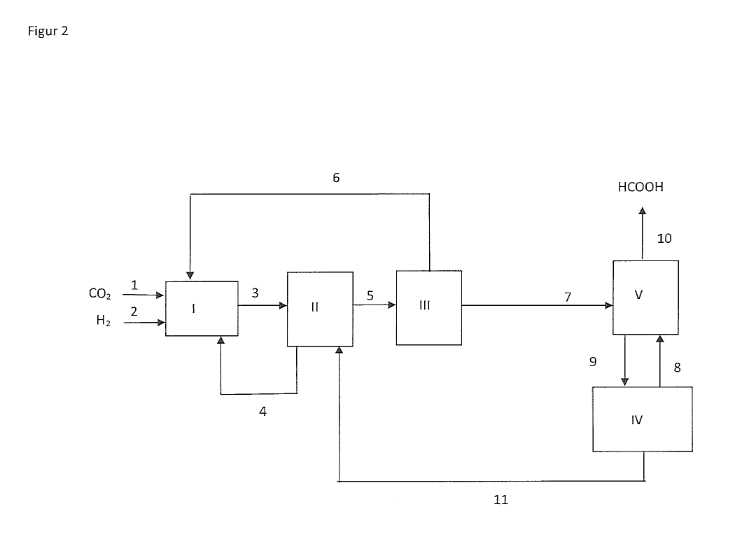

FIG. 2 shows a block diagram of a preferred embodiment of the process of the invention,

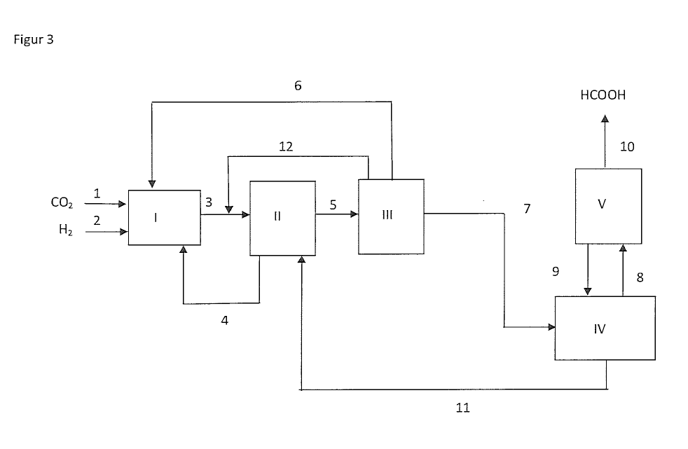

FIG. 3 shows a block diagram of a further preferred embodiment of the process of the invention,

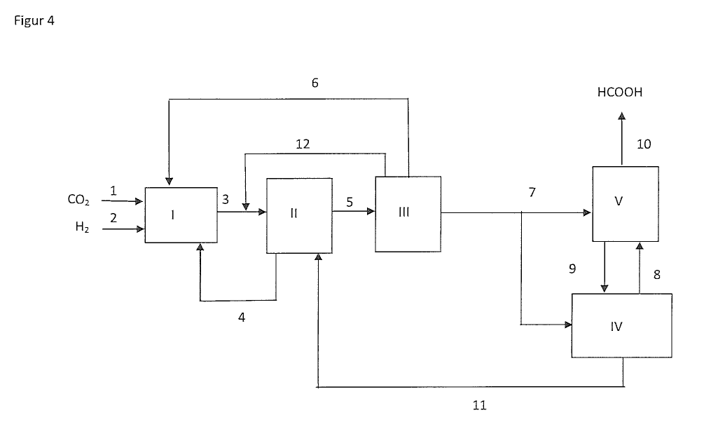

FIG. 4 shows a block diagram of a preferred embodiment of the process of the invention,

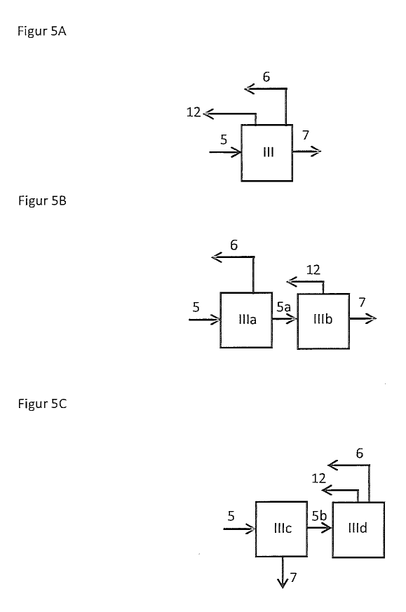

FIGS. 5A, 5B and 5C each show different, preferred variants for the fractional removal of polar solvent and water for the preferred embodiment in FIG. 3.

In the embodiment shown in FIG. 1, carbon dioxide, stream (1) and hydrogen, stream (2), are fed into the hydrogenation reactor I. In this reactor, they are reacted in the presence of a catalyst comprising an element of group 8, 9 or 10 of the Periodic Table, a tertiary amine and a polar solvent to form formic acid-amine adducts. The output from the hydrogenation reactor (I) (stream (3)), which can consist of one or two phases, is fed to the extraction unit II in which the catalyst is extracted by means of the tertiary amine from the phase separation vessel (IV) (stream (11)). The tertiary amine with the catalyst (stream (6)) from the extraction unit (II) is recirculated to the hydrogenation reactor (I). The product phase (5) from the extraction unit (II) is fed to the thermal separation unit (III) in order to separate off the polar solvent and the water thermally from the formic acid-amine adducts. The stream (5) can additionally be passed over an adsorber bed to remove last traces of catalyst from this stream before the thermal separation. The polar solvent which is thermally separated off in the distillation unit III is recirculated as stream (6) to the hydrogenation reactor (I) and the two-phase bottoms mixture (stream 7) from the distillation unit (III), which comprises the formic acid-amine adducts and the tertiary amine, is fed to the phase separation vessel (IV). The formic acid-amine adducts are separated off in the phase separation vessel (IV) and fed as stream (8) to the distillation unit (V) in which they are thermally dissociated into free formic acid and tertiary amine. The free formic acid is taken off as overhead product, stream (10), and the two-phase bottoms from the distillation unit (V), which comprise tertiary amine and undissociated formic acid/amine adducts, stream (9) are fed back into the phase separation vessel (IV). The tertiary amine which is separated off in the phase separation vessel (IV) is fed as stream (11) to the extraction unit (II) in order to extract the catalyst, or parts of stream (11) can be recirculated directly to the hydrogenation reactor (I) if not all the trialkylamine is required for the extraction.

In the embodiment shown in FIG. 2, carbon dioxide, stream (1), and hydrogen, stream (2), are introduced into the hydrogenation reactor (I). In this reactor, they are reacted in the presence of a catalyst comprising an element of group 8, 9 or 10 of the Periodic Table, a tertiary amine and a polar solvent to form formic acid-amine adducts. The output from the hydrogenation reactor (I) (stream (3)), which can consist of one or two phases, is fed to the extraction unit II in which the catalyst is extracted by means of the tertiary amine from the phase separation vessel (IV) (stream (11)). The tertiary amine with the catalyst (stream (6)) from the extraction unit (II) is recirculated to the hydrogenation reactor (I). The product phase (5) from the extraction unit (II) is fed to the thermal separation unit (III) in order to separate off the polar solvent and the water thermally from the formic acid-amine adducts. The stream (5) can be passed over an adsorber bed to remove last traces of catalyst from this stream before the thermal separation. The polar solvent which is separated off thermally in the distillation unit III is recirculated as stream (6) to the hydrogenation reactor (I) and the two-phase bottoms mixture from the distillation unit (III), which comprises the formic acid-amine adducts and the tertiary amine (stream 7), is fed to the distillation unit (V). In this distillation unit, the formic acid/amine adducts are thermally dissociated into free formic acid and tertiary amine. The free formic acid is, for example, removed as overhead product, stream (10). The two-phase bottoms from the distillation unit (V), stream (9), which comprise tertiary amine and undissociated formic acid/amine adducts, are fed to the phase separation vessel (IV). The tertiary amine which is separated off in the phase separation vessel (IV) is fed as stream (11) to the extraction unit (II). Part of stream (11) can also be recirculated directly to the hydrogenation reactor (I) if not all the amine is required for the extraction.

In the embodiment shown in FIG. 3, carbon dioxide, stream (1), and hydrogen, stream (2), are introduced into the hydrogenation reactor (I). In this reactor, they are reacted in the presence of a catalyst comprising an element of group 8, 9 or 10 of the Periodic Table, a tertiary amine and a polar solvent to form formic acid-amine adducts. The output from the hydrogenation reactor (I) (stream (3)), which can consist of one or two phases, is admixed with the predominantly water-comprising stream (12) from the distillation vessel (III) in order to obtain better phase separation and catalyst distribution in the extraction together with high hydrogenation activity. This stream is fed to the extraction unit (II) in which the catalyst is extracted by means of the tertiary amine from the phase separation vessel (IV) (stream (11)). The tertiary amine with the catalyst (stream (6)) from the extraction unit (II) is recirculated to the hydrogenation reactor (I), The product phase (5) from the extraction unit (II) is fed to the thermal separation unit (III) in order to separate off the polar solvent and the water thermally from the formic acid-amine adducts. The stream (5) can be passed over an adsorber bed to remove last traces of catalyst from this stream before the thermal separation. The polar solvent which is separated off thermally in the distillation unit III is recirculated as stream (6) to the hydrogenation reactor I, the water is recirculated as stream (12) to the output from the hydrogenation reactor (stream (3)) and the two-phase bottoms mixture from the distillation unit III, which comprises the formic acid-amine adducts and the tertiary amine (stream (7)), is fed to the phase separation vessel (IV). The formic acid-amine adducts are separated off in the phase separation vessel (IV) and fed as stream (8) to the distillation unit (V) in which they are thermally dissociated into free formic acid and tertiary amine. The free formic acid is taken off as overhead product, stream (10), and the two-phase bottoms from the distillation unit (IV), which comprise tertiary amine and undissociated formic acid/amine adducts, stream (9), are fed back into the phase separation vessel (IV). The tertiary amine which is separated off in the phase separation vessel (IV) is fed as stream (11) to the extraction unit (II) in order to extract the catalyst or part of stream (11) can be recirculated directly to the hydrogenation reactor (I) if not all the trialkylamine is required for the extraction.

The embodiment shown in FIG. 4 differs from the embodiments in FIGS. 1 and 2 in that part of the two-phase bottoms mixture (stream (7)) is fed to the phase separation vessel IV and part of the two-phase bottoms mixture (stream (7)) is fed to the distillation unit (V).

FIGS. 5A, 5B and 5C each schematically show different variants for the thermal removal of water and polar solvent for the embodiment shown in FIG. 3.

Here, FIG. 5A shows the removal of two streams, i.e. the stream (6) comprising predominantly polar solvent and the stream (12) comprising predominantly water, in a single distillation column which can be a column having a side offtake or a dividing wall column.

FIG. 5B schematically shows an embodiment with a 2-column variant (columns (IIIa) and (IIIb)), where the lower-boiling component, generally the polar solvent, is separated off in the first column, IIIa, and the intermediate-boiling component, generally the water, is separated off as stream (12) in the second column, (IIIb), to which a feed stream (5a) depleted in polar solvent is fed.

FIG. 5C shows a further embodiment for the removal of water and polar solvent, in which the phase (7) comprising the formic acid/amine adducts and the tertiary amine is firstly separated off in a first distillation column (IIIc) and a stream (5b) is subsequently fractionated in a second distillation column (IIId) to give a stream (6) comprising predominantly polar solvent and a stream (12) comprising predominantly water.

1. A process for preparing formic acid by reacting carbon dioxide (1) with hydrogen (2) in a hydrogenation reactor (I) in the presence of
a catalyst comprising an element of group 8, 9 or 10 of the Periodic Table,
a tertiary amine comprising at least 12 carbon atoms per molecule and
a polar solvent comprising one or more monoalcohols selected from among methanol, ethanol, propanols and butanols,
to form formic acid/amine adducts as intermediates which are subsequently thermally dissociated,
where a tertiary amine having a boiling point which is at least 5° C. higher than that of formic acid is used and
a reaction mixture comprising the polar solvent, the formic acid/amine adducts, the tertiary amine and the catalyst is formed in the reaction in the hydrogenation reactor (I) and is discharged from the reactor as output (3),
wherein
the output (3) from the hydrogenation reactor (I) is, optionally after addition of water, fed directly to an extraction unit (II) and the work-up of the output (3) comprises the following process steps:
1) extraction of the catalyst from the output (3) from the hydrogenation reactor (I) in the extraction unit (II), where the same tertiary amine which was used in the hydrogenation is used as extractant, to give an extract (4) which comprises the major part of the tertiary amine and the catalyst and is recycled to the hydrogenation reactor (I) and also a raffinate (5) which comprises the major part of the polar solvent and the formic acid/amine adducts and is passed on to a distillation unit (III) in which
2) a distillation is carried out to give an overhead stream (6) which comprises predominantly the polar solvent and is recycled to the hydrogenation reactor (I) and also a bottom stream (7) which comprises predominantly the formic acid/amine adducts and the tertiary amine and is according to process step
3) fed to a phase separation vessel (IV) in which
a) a phase separation is carried out to give an upper phase which comprises predominantly the tertiary amine and is recycled as stream (11) to the extraction unit (II) as extractant for the catalyst and a lower phase which comprises predominantly the formic acid/amine adducts and is fed as stream (8) to a thermal dissociation unit (V) in which
b) a thermal dissociation is carried out to give a stream (9) which comprises the tertiary amine and is recirculated to the phase separation vessel (IV) and a stream (10) comprising the formic acid
or according to process step
4) is fed to a thermal dissociation unit (V) in which
a) a thermal dissociation is carried out to give a stream (10) comprising the formic acid and a stream (9) which comprises the tertiary amine and the formic acid/amine adducts and is fed to a phase separation vessel (IV) in which
b) a phase separation is carried out to give an upper phase which comprises predominantly the tertiary amine and is recycled as stream (11) to the extraction unit (II) as extractant for the catalyst and a lower phase which comprises predominantly the formic acid/amine adducts and is fed as stream (8) to a thermal dissociation unit (V).

2. The process according to claim 1, wherein a substream of the bottom stream (7) is worked up as per process step 3) and the remainder of the bottom stream (7) is worked up as per process step 4).

3. The process according to claim 1 or 2, wherein, in process step 2), a fractional distillation is carried out to give a first fraction (6) which comprises predominantly the polar solvent and is recycled to the hydrogenation reactor (I) and a second fraction (12) which comprises predominantly water and is recycled to the output (3) from the hydrogenation reactor (I).

4. The process according to claim 1, wherein an amine of the general formula (Ia)
NR¹R²R³ (Ia)
where the radicals R¹ to R³ are identical or different and are each, independently of one another, an unbranched or branched, acyclic or cyclic, aliphatic, araliphatic or aromatic radical having in each case from 1 to 6 carbon atoms, where individual carbon atoms can also, independently of one another, be substituted by a heterogroup selected from the group consisting of —O— and >N— and two or all three of the radicals can also be joined to one another to form a chain comprising at least four atoms, with the proviso that the tertiary amine comprises at least 12 carbon atoms per molecule, is used as tertiary amine.

5. The process according to claim 4, wherein an amine of the general formula (Ia) in which the radicals R¹ to R³ are selected independently from the group consisting of C₁-C₁₂-alkyl, C₅-C₈-cycloalkyl, benzyl and phenyl is used as tertiary amine.

6. The process according to claim 4, wherein a saturated amine of the general formula (Ia) is used as tertiary amine.

7. The process according to claim 4, wherein an amine of the general formula (la) in which the radicals R¹ to R³ are selected independently from among C₅- and C₆-alkyl is used as tertiary amine.

8. The process according to claim 1, wherein a mixture of one or more monoalcohols selected from among methanol, ethanol, propanols and butanols with water is used as polar solvent.

9. The process according to claim 1, wherein a mixture of methanol and/or ethanol with water is used as polar solvent.

10. The process according to claim 1, wherein methanol and/or ethanol is used as polar solvent.

11. The process according to claim 1, wherein the raffinate (5) obtained in process step 1) has a water content, based on the total weight of the raffinate, in the range from 0.1 to 50% by weight, preferably in the range from 2 to 30% by weight.

12. The process according to claim 1, wherein the catalyst is a homogeneous catalyst.

13. The process according to claim 9, wherein the homogeneous catalyst is a complex comprising an element of group 8, 9 or 10 of the Periodic Table and at least one phosphine group having at least one unbranched or branched, acyclic or cyclic aliphatic radical having from 1 to 12 carbon atoms, where individual carbon atoms can also be substituted by >P—.

14. The process according to claim 1, wherein the reaction in the hydrogenation reactor (I) is carried out at a temperature in the range from 20 to 200° C. and a pressure in the range from 0.2 to 30 MPa abs.

15. The process according to claim 1, wherein the extraction is carried out at temperatures in the range from 40 to 80° C.
